(19) United States
(12) Patent Application Publication
(10) Pub. No.: US 2002/0007079 A1
(43) Pub. Date: Jan. 17, 2002

(54) MEIOSIS REGULATING COMPOUNDS

(76) Inventors: ANTHONY MURRAY, HELLERUP (DK); PETER FAARUP, VAERLOSE (DK)

Correspondence Address: STEVE T ZELSON ESQ, NOVO NORDISK OF NORTH AMERICA INC, 405 LEXINGTON AVENUE, SUITE 6400, NEW YORK, NY, 101746400

(21) Appl. No.: 09/350,193

(22) Filed: Jul. 9, 1999

Related U.S. Application Data
(63) Continuation of application No. PCT/DK99/00333, filed on Jun. 18, 1999.
(60) Provisional application No. 60/092,763, filed on Jul. 14, 1998.
(60) Provisional application No. 60/093,025, filed on Jul. 16, 1998.

(30) Foreign Application Priority Data
Jun. 19, 1998 (DK) PA 199800807
Jun. 19, 1998 (DK) PA 1998 00810
Feb. 4, 1999 (DK) PA 1999 00140
Feb. 4, 1999 (DK) PA 1999 00141

(51) Int. Cl. C07J 1/00; C07J 9/00

(52) U.S. Cl. 552/540; 552/541; 552/542; 552/543; 552/546; 552/544; 552/508

(57) ABSTRACT

Certain novel sterol derivatives having no hydroxy group in the 3-position can be used to regulate the meiosis in oocytes and in male germ cells. Some of these compounds are useful in the treatment of infertility, whereas other compounds are useful as contraceptives. These compounds have the structural formula

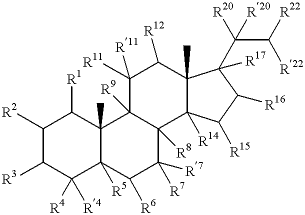

wherein the substituents are as defined in the specification.

### CROSS-REFERENCE TO RELATED APPLICATIONS

This application is a continuation application of PCT/DK99/00333 filed on Jun. 18, 1999 and claims priority under 35 U.S.C. 119 of Danish application nos. PA 1998 00807 filed Jun. 19, 1998, PA 1998 00810 filed Jun. 19, 1998, PA 1999 00140 filed Feb. 4, 1999, PA 1999 00141 filed Feb. 4, 1999 and U.S. provisional application Nos. 60/092,763 filed Jul. 14, 1998 and 60/093,025 filed Jul. 16, 1998, the contents of which are fully incorporated herein by reference.

### FIELD OF THIS INVENTION

The present invention relates to certain novel pharmacologically active compounds, to novel pharmaceutical compositions containing certain compounds as active substance and to the novel use of certain compounds as medicaments. More particularly, it has been found that the compounds described herein can be used for regulating the meiosis.

### BACKGROUND OF THIS INVENTION

Meiosis is the unique and ultimate event of germ cells on which sexual reproduction is based. Meiosis comprises two meiotic divisions. During the first division, exchange between maternal and paternal genes take place before the pairs of chromosomes are separated into the two daughter cells. These contain only half the number (1 n) of chromosomes and 2c DNA. The second meiotic division proceeds without a DNA synthesis. This division therefore results in the formation of the haploid germ cells with only 1 c DNA.

The meiotic events are similar in the male and female germ cells, but the time schedule and the differentiation processes which lead to ova and to spermatozoa differ profoundly. All female germ cells enter the prophase of the first meiotic division early in life, often before birth, but all are arrested as oocytes later in the prophase (dictyate state) until ovulation after puberty. Thus, from early life the female has a stock of oocytes which is drawn upon until the stock is exhausted. Meiosis in females is not completed until after fertilization, and results in only one ovum and two abortive polar bodies per germ cell. In contrast, only some of the male germ cells enter meiosis from puberty and leave a stem population of germ cells throughout life. Once initiated, meiosis in the male cell proceeds without significant delay and produces 4 spermatozoa.

Only little is known about the mechanisms which control the initiation of meiosis in the male and in the female. In the oocyte, new studies indicate that follicular purines, hypoxanthine or adenosine, could be responsible for meiotic arrest (Downs, S. M., et al. in Dev Biol. 82 (1985), 454-458; Eppig, J. J., et al. in Dev Biol. 119 (1986), 313-321; and Downs, S. M., in Mol. Reprod.Dev. 35 (1993), 82-94). The presence of a diffusible meiosis regulating substance was first described by Byskov et al. in a culture system of fetal mouse gonads (Byskov, A. G. et al. in Dev Biol. 52 (1976), 193-200). A meiosis activating substance (MAS) was secreted by the fetal mouse ovary in which meiosis was ongoing, and a meiosis preventing substance (MPS) was released from the morphologically differentiated testis with resting, non-meiotic germ cells. It was suggested that the relative concentrations of MAS and MPS regulated the beginning, arrest and resumption of meiosis in the male and in the female germ cells (Byskov, A. G. et al. in The Physiology of Reproduction (editors: Knobil, E., and Neill, J. D., Raven Press, New York (1994)). Clearly, if meiosis can be regulated, reproduction can be controlled. In Nature 374 (1995), 559-562, Byskov et al. describes the isolation from bull testes and from human follicular fluid of certain sterols that activate oocyte meiosis. Unfortunately, these sterols are rather labile and utilization of the interesting finding would thus be greatly facilitated if more stable meiosis activating compounds were available.

Biochim.Biophys.Acta 1299 (1996), 313, deals with mechanism and structural requirements for transformation of substrates by a specific transferase and mentions, e.g., cholest-4,8,24-triene and 25-azacholest-5-ene (compounds 21 & 42 in FIG. 2 ).

Bull.Chem.Soc.Belg. 92 (1983), 731, deals with magnetic resonance spectra of some steroids, e.g., of cholestane (compound g in Table V).

Collect.Czech.Chem.Comm. 63 (1998), 549, deals with preparation of some steroids, e.g., of cholest-3,5-diene; cholest-2-ene; and cholest-5-ene (compounds 2, 5 & 7).

Environ.Sci.Tech. 23 (1989), 688, deals with chemical composition of environmental tobacco smoke and mentions, e.g., cholesta-3,5-diene; 24-methylcholesta-3,5-diene; 24-ethylcholesta-3,5,22-diene; and 24-ethylcholest-3,5-diene (compounds e, f, g & h in FIG. 6 ).

Geochim.Cosmochim. 51 (1987), 3051, deals with steroid geochemistry in the oxygen minimum zone of the eastern tropical North Pacific Ocean and mentions, e.g., cholest-2-ene and cholest-3,5-diene (compounds 5 & 8 in Table 6).

Geochim.Cosmochim. 55 (1991), 1065, deals with analysis and occurrence of C₂₆-steranes in petroleum and source rocks and mentions, e.g., 24-nor-5α-cholestane and 24-nor-5β-cholestane (compounds 1Bb & 1Ba in FIG. 3 ).

Geochim.Cosmochim.Acta 57 (1993), 4539, deals with norcholestane in Miocene Onnagawa siliceous sediments in Japan and mentions, e.g., (20R)-5β,14α,17α(H)-cholestane; (20R)-5α,14β,17β(H)-cholestane; (20R)-5α,14α,17α(H)-cholestane; (20R)-5β,14α,17α(H)-24-methylcholestane; (20R)-5α,14α,17α(H)-24-methylcholestane; (20R)-5β,14α,17α(H)-24-ethylcholestane; and (20R)-5α,14α,17α(H)-24-ethylcholestane (peaks 3a, 3b, 6, 8, 10, 12 & 13).

Initial Reports of the Deep Sea Drilling Project 62, 923, deals with lipids of upper albian limestone and mentions, e.g., 4-methyl-5α-24-norcholestane; 5α-cholestane; 5β-cholestane; cholest-4-ene; cholest-5-ene; and 4-methylcholestane (compounds L, O & N in Table 1, compounds XIVa & XVa in Table 3 and compound 9 in Table 14).

Initial Reports of the Deep Sea Drilling Project 63, 763, deals with preliminary lipid analysis of sediments from the eastern North Pacific Ocean and mentions, e.g., (20S)-5α,14α,17α-cholestane; (20R)-5α,14α,17α-cholestane; 19-nor-5α-cholestane; 5β-cholestane; 5α-cholestane; cholest-2-ene; cholesta-3,5-diene; cholest-4-ene; and cholest-5-ene (compounds VIIi & VIIj in Table 1, compounds XVj, VIIj & VIIj in Table 2, compounds XIj & XIVj in Table 3 and compounds XIIj & XIIIj in Table 5).

Initial Reports of the Deep Sea Drilling Project 63, 837, deals with organic geo-chemistry of sediments from the southern California borderland and mentions, e.g., nor-cholestane; 5α,8β,14β-cholestane; and 5β,8β,14β-cholestane (compounds IX & X).

Initial Reports of the Deep Sea Drilling Project 64, 837, deals with organic petrography and extractable hydrocarbons of sediment from the gulf of California and mentions, e.g., 5α-norcholestane; 5β-cholestane; cholest-4-ene; cholest-5-ene; and 5α-cholestane (peaks e, f, h, i & j in Table 2).

J.Chromatog. 116 (1976), 207, deals with chromatography of saturated steroid hydrocarbons on alumina and mentions, e.g., 5β-cholestane; 5α,14β-cholestane; 5α,17β(H)-cholestane; (20S)-5α,17β(H)-cholestane; (24R)-24-methyl-5β-cholestane; (24S)-24-methyl-5β-cholestane; 5α,8α,14β-cholestane; (20S)-5α-cholestane; (24R)-24-methyl-5α-cholestane; (24S)-24-ethyl-5α-cholestane; (24S)-24-ethyl-5α-cholestane; 5α-cholestane; 4α-methyl-5α-cholestane; 4β-methyl-5α-cholestane; and (24S)-24-methyl-5α-cholestane (Table I).

J.Org.Chem. 37 (1972), 2108, deals with the chemistry of a diazo ketone and its derivatives obtained from cholanic acid and mentions, e.g., 24-hydroxymethylchola-24-one and 24-hydroxymethylchola-24-ol (compounds 6 & 12).

Marine and Petroleum Geology 5 (1988), 205, deals with geochemical and biological marker assessment of depositional environments using Brazilian offshore oils and mentions, e.g., (20S)-5α,14α,17α-cholestane and (20R)-5α,14α,17α-cholestane (compounds 8 & 10).

OPPI Briefs, 16, deals with the synthesis of sterols with modified side chain by Wittig reaction and mentions, e.g., cholest-24-ene and 24-cyclohexylchola-24-ene (compounds V & VI).

Org.Geochem. 9 (1986), 331, deals with lipid composition of a crab, its feces, and sinking particulate organic matter in the Equatorial North Pacific Ocean and mentions, e.g., cholest-2-ene; cholesta-3,5-diene; 24-methylcholest-2-ene; and 24-ethylcholest-2-ene (compounds 2, 5, 9 & 15 in Table 1).

Org.Geochem. 19 (1991), 351, deals with structural investigations of sulphur-rich macromolecular oil fractions and a kerogen by sequential chemical degradation and mentions, e.g., 24-propylcholestane (FIG. 15 ).

In a publication by C. Djerassi about Rearrangement Reactions in Organic Mass Spectroscopy, 199, e.g., 5α-cholestane (FIG. 1 ) is mentioned.

Steroids 18 (1971), 649, deals with steroidal triphenyl salts, versatile intermediates for side chain modifications, and mentions, e.g., cholest-25-ene and 24-cyclohexylchola-24-ene (compounds 4 & 5).

Tetrahedron Letters 22 (1981), 2583, deals with dissolving metal reduction by crown ether and mentions, e.g., 5α-cholestane and cholest-5-ene (compounds 3 & 6).

Tetrahedron Letters 34 (1973), 3175, deals with identification of C₂₄ alkylated steranes by P.M.R. spectroscopy and mentions, e.g., 5α-cholestane and 24-dimethyl-5α-cholane (compounds 1 & 2).

In the last mentioned 21 publications, we have found no statement about pharmacological properties of the specific compounds cited from said publications.

Compounds being known to stimulate the meiosis and being different from the compounds claimed in the present patent application are described in international patent applications Nos. WO 96/00235, 96/27658, 97/00884, 98/52965 and 98/55498.

The compounds described herein have advantages compared with the known compounds.

### SUMMARY OF THE INVENTION

A main purpose of this invention is to furnish compounds which can be used to regulate meiosis.

One purpose of the present invention is to provide compounds and methods useful for relieving infertility in females and males, particularly in mammals, more particularly in humans.

In a further object, the present invention concerns the use of the compounds of the general formula Ib (stated in the claims, below) and esters, salts, active metabolites and pro-drugs thereof for relieving infertility in females and males, particularly in mammals, more particularly in humans.

In still another preferred embodiment, the present invention relates to compounds of the general formula Ib and esters, salts, active metabolites and prodrugs thereof as a medicament.

In a further preferred embodiment, this invention relates to compounds of the general formula Ib or esters, salts, active metabolites and prodrugs thereof in the manufacture of a medicament for use in the regulation of meiosis.

In a further preferred aspect, the present invention relates to the use of a compound of formula Ib above or an ester, salt, active metabolite and prodrug thereof as a medicament, in particular as a medicament for use in the regulation of meiosis. The compound may be used neat or in the form of a liquid or solid composition containing auxiliary ingredients conventionally used in the art.

In the present context, the expression “regulating the meiosis” is used to indicate that certain of the compounds of formula Ia and Ib can be used for stimulating the meiosis in vitro, in vivo, or ex vivo. Thus, the compounds which may be agonists of a naturally occurring meiosis activating substance, can be used in the treatment of infertility which is due to insufficient stimulation of meiosis in females and in males. Other compounds of formula Ia and Ib, which may be antagonists of a naturally occurring meiosis activating substance, can be used for regulating the meiosis, preferably in vivo, in a way which makes them suited as contraceptives. In this case the “regulation” means partial or total inhibition.

In a still further preferred aspect, the present invention relates to the use of a compound of formula Ib above or an ester, salt, active metabolite and prodrug thereof in the regulation of the meiosis of an oocyte, in particular a mammalian oocyte, more particularly a human oocyte.

In a still further preferred aspect, the present invention relates to the use of a compound of formula Ib above or an ester, salt, active metabolite and prodrug thereof in the stimulation of the meiosis of an oocyte, in particular a mammalian oocyte, more particularly a human oocyte.

In a still further preferred aspect, the present invention relates to the use of a compound of formula Ib above or an ester, salt, active metabolite and prodrug thereof in the inhibition of the meiosis of an oocyte, in particular a mammalian oocyte, more particularly a human oocyte.

In a still further preferred aspect, the present invention relates to the use of a compound of formula Ib above or an ester, salt, active metabolite and prodrug thereof in the regulation of the meiosis of a male germ cell, in particular a mammalian male germ cell, more particularly a human male germ cell.

In a still further preferred aspect, the present invention relates to the use of a compound of formula Ib above or an ester, salt, active metabolite and prodrug thereof in the stimulation of the meiosis of a male germ cell, in particular a mammalian male germ cell, more particularly a human male germ cell.

In a still further preferred aspect, the present invention relates to the use of a compound of formula Ib above or an ester, salt, active metabolite and prodrug thereof in the inhibition of the meiosis of a male germ cell, in particular a mammalian male germ cell, more particularly a human male germ cell.

In a yet still further preferred aspect, the present invention relates to a method of regulating the meiosis in a mammalian germ cell which method comprises administering an effective amount of a compound of formula Ib above or an ester, salt, active metabolite and prodrug thereof to a germ cell in need of such a treatment.

In a still further aspect, the present invention relates to a method of regulating the meiosis in a mammalian germ cell wherein a compound of formula Ib above or an ester, salt, active metabolite and prodrug thereof is administered to the germ cell by administering the compound to a mammal hosting said cell.

In a still further aspect, the present invention relates to a method wherein the germ cell the meiosis of which is to be regulated by means of a compound of formula Ib above or an ester, salt, active metabolite and prodrug thereof is an oocyte.

In a still further aspect, the present invention relates to a method of regulating the meiosis in an oocyte wherein a compound of formula Ib above or an ester, salt, active metabolite and prodrug thereof is administered to the oocyte ex vivo.

In a still further aspect, the present invention relates to a method of regulating the meiosis of a male germ cell by administering a compound of formula Ib above or an ester, salt, active metabolite and prodrug thereof to the cell.

In a still further aspect, the present invention relates to a method whereby mature male germ cells are produced by administering in vivo or in vitro a compound of formula Ib above or an ester, salt, active metabolite and prodrug thereof to testicular tissue containing immature cells.

In a still further aspect, the present invention relates to compounds having superior in vitro properties.

### DETAILED DESCRIPTION OF THIS INVENTION

According to the present invention there are provided compounds with interesting pharmacological properties. The compounds described herein are useful for regulating the meiosis in oocytes and in male germ cells.

It has surprisingly been found that compounds of formula Ib having no hydroxy group in the 3-position have favorable action in the regulation of meiosis. One reason this is surprising is that a 3-hydroxy group is present in the natural cholesterol and in the compounds participating in the biosynthesis thereof, including 4,4-dimethyl-5α-cholesta-8,14,24-triene-3β-ol (hereinafter designated FF-MAS) and 4,4-dimethyl-5β-choleste-8,24-diene-3β-ol.

Preferred compounds of formula Ia and Ib are such having at least one double bond.

Other preferred compounds of formula Ia and Ib are such wherein R¹ is hydrogen.

Other preferred compounds of formula Ia and Ib are such wherein R¹ is halogen.

Other preferred compounds of formula Ia and Ib are such wherein R¹ is methyl.

Other preferred compounds of formula Ia and Ib are such wherein R¹ is hydroxy

Other preferred compounds of formula Ia and Ib are such wherein R¹ is oxo.

Other preferred compounds of formula Ia and Ib are such wherein R², together with

R³, designates an additional bond between the carbon atoms at which R² and R³ are placed.

Other preferred compounds of formula Ia and Ib are such wherein R² is hydrogen.

Other preferred compounds of formula Ia and Ib are such wherein R² is hydroxy.

Other preferred compounds of formula Ia and Ib are such wherein R² is C₁-₃ alkyl.

Other preferred compounds of formula Ia and Ib are such wherein R² is C₁-C₃ alkoxy.

Other preferred compounds of formula Ia and Ib are such wherein R²is halogen.

Other preferred compounds of formula Ia and Ib are such wherein R³is hydrogen.

Other preferred compounds of formula Ia and Ib are such wherein R³is C₁-C₄ alkyl.

Preferred compounds of formula Ia and Ib are such wherein R⁴ and R′⁴ are both hydrogen.

Other preferred compounds of formula Ia and Ib are such wherein one of R⁴ and R′⁴ is hydrogen while the other is methyl.

Other preferred compounds of formula Ia and Ib are such wherein R⁴ and R′⁴ are both methyl.

Other preferred compounds of formula Ia and Ib are such wherein R⁴ is branched or unbranched C₁-₆ alkyl, optionally substituted by halogen, hydroxy or cyano.

Other preferred compounds of formula Ia and Ib are such wherein R′⁴ is branched or unbranched C₁-C₆ alkyl, optionally substituted by halogen, hydroxy or cyano.

Other preferred compounds of formula Ia and Ib are such wherein R⁴ is hydroxy and R′⁴ is selected from the group comprising hydrogen and branched or unbranched C₁-C₆alkyl which may be substituted by halogen, hydroxy or cyano.

Other preferred compounds of formula Ia and Ib are such wherein R⁴ and R′⁴ together designate methylene.

Other preferred compounds of formula Ia and Ib are such wherein R⁴ and R′⁴, together with the carbon atom to which they are bound, form a cyclopropane ring.

Other preferred compounds of formula Ia and Ib are such wherein R⁴ and R′⁴, together with the carbon atom to which they are bound, form a cyclopentane ring.

Other preferred compounds of formula Ia and Ib are such wherein R⁴ and R′⁴, together with the carbon atom to which they are bound, form a cyclohexane ring.

Other preferred compounds of formula Ia and Ib are such wherein R⁵ is hydrogen.

Other preferred compounds of formula Ia and Ib are such wherein R⁵ is halogen.

Other preferred compounds of formula Ia and Ib are such wherein R⁵is hydroxy.

Other preferred compounds of formula Ia and Ib are such wherein R⁶ is hydrogen.

Other preferred compounds of formula Ia and Ib are such wherein R⁶ is halogen.

Other preferred compounds of formula Ia and Ib are such wherein R⁶ is oxo.

Other preferred compounds of formula Ia and Ib are such wherein R⁶ is hydroxy.

Other preferred compounds of formula Ia and Ib are such wherein R⁶, together with R⁵ designates an additional bond between the carbon atoms at which R⁵ and R⁶ are placed.

Other preferred compounds of formula Ia and Ib are such wherein R⁷ is hydrogen.

Other preferred compounds of formula Ia and Ib are such wherein R⁷ and R′⁷ together are methylene.

Other preferred compounds of formula Ia and Ib are such wherein R⁷ is hydroxy.

Other preferred compounds of formula Ia and Ib are such wherein R⁷ is methoxy or acetoxy.

Other preferred compounds of formula Ia and Ib are such wherein R⁷ is halogen.

Other preferred compounds of formula Ia and Ib are such wherein R⁷ and R′⁷ together are oxo.

Other preferred compounds of formula Ia and Ib are such wherein R⁷ and R′⁷ together are the group=NOH.

Other preferred compounds of formula Ia and Ib are such wherein R⁷ and R′⁷ together are a group of the general formula=NOR³⁶, wherein R³⁶ is C₁-₃ alkyl.

Other preferred compounds of formula Ia and Ib are such wherein R⁷ is hydroxy and R′⁷ is C₁-₄ alkyl.

Other preferred compounds of formula Ia and Ib are such wherein R⁷, together with R⁶, designates an additional bond between the carbon atoms at which R⁷ and R⁶ are placed.

Other preferred compounds of formula Ia and Ib are such wherein R⁷, together with R⁸, designates an additional bond between the carbon atoms at which R⁷ and R⁸ are placed.

Other preferred compounds of formula Ia and Ib are such wherein R⁸, together with R⁹, designates an additional bond between the carbon atoms at which R⁸ and R⁹ are placed.

Other preferred compounds of formula Ia and Ib are such wherein R⁸ is hydrogen.

Other preferred compounds of formula Ia and Ib are such wherein R⁸ is halogen.

Other preferred compounds of formula Ia and Ib are such wherein R⁸ is hydroxy.

Other preferred compounds of formula Ia and Ib are such wherein R⁹ is hydrogen.

Other preferred compounds of formula Ia and Ib are such wherein R⁹ is halogen.

Other preferred compounds of formula Ia and Ib are such wherein R⁹ is hydroxy.

Other preferred compounds of formula Ia and Ib are such wherein R¹¹ is hydrogen.

Other preferred compounds of formula Ia and Ib are such wherein R¹¹ and R′¹¹ together are methylene.

Other preferred compounds of formula Ia and Ib are such wherein R¹¹ is hydroxy.

Other preferred compounds of formula Ia and Ib are such wherein R¹¹ is halogen.

Other preferred compounds of formula Ia and Ib are such wherein R¹¹ is methoxy or acetoxy.

Other preferred compounds of formula Ia and Ib are such wherein R¹¹ and R′¹¹ together are oxo.

Other preferred compounds of formula Ia and Ib are such wherein R¹¹ and R′¹¹ together are the group=NOH.

Other preferred compounds of formula Ia and Ib are such wherein R¹¹ and R′¹¹ together are a group of the general formula=NOR³⁷, wherein R³⁷ is C₁-₃ alkyl.

Other preferred compounds of formula Ia and Ib are such wherein R¹¹ is hydroxy and R′¹¹ is C₁-C₄ alkyl.

Other preferred compounds of formula Ia and Ib are such wherein R¹¹, together with R⁹, designates an additional bond between the carbon atoms at which R¹¹ and R⁹ are placed.

Other preferred compounds of formula Ia and Ib are such wherein R¹¹, together with R¹², designates an additional bond between the carbon atoms at which R¹¹ and R¹² are placed.

Other preferred compounds of formula Ia and Ib are such wherein R¹² is hydrogen.

Other preferred compounds of formula Ia and Ib are such wherein R¹² is halogen.

Other preferred compounds of formula Ia and Ib are such wherein R¹² is C₁-C₄ alkyl.

Other preferred compounds of formula Ia and Ib are such wherein R¹² is methylene.

Other preferred compounds of formula Ia and Ib are such wherein R¹² is hydroxy.

Other preferred compounds of formula Ia and Ib are such wherein R¹² is methoxy or acetoxy.

Other preferred compounds of formula Ia and Ib are such wherein R¹² is oxo.

Other preferred compounds of formula Ia and Ib are such wherein R¹² is the group=NOH.

Other preferred compounds of formula Ia and Ib are such wherein R¹² is a group of the general formula=NOR³³, wherein R³³ is C₁-C₃ alkyl.

Other preferred compounds of formula Ia and Ib are such wherein R¹⁴ is hydrogen.

Other preferred compounds of formula Ia and Ib are such wherein R¹⁴ is hydroxy.

Other preferred compounds of formula Ia and Ib are such wherein R⁴, together with R⁸, designates an additional bond between the carbon atoms at which R¹⁴ and R⁸ are placed.

Other preferred compounds of formula Ia and Ib are such wherein R¹⁵ is hydrogen.

Other preferred compounds of formula Ia and Ib are such wherein R¹⁵ is halogen.

Other preferred compounds of formula Ia and Ib are such wherein R¹⁵ is C₁-C₄ alkyl.

Other preferred compounds of formula Ia and Ib are such wherein R¹⁵ is methylene.

Other preferred compounds of formula Ia and Ib are such wherein R¹⁵ is hydroxy.

Other preferred compounds of formula Ia and Ib are such wherein R¹⁵ is methoxy.

Other preferred compounds of formula Ia and Ib are such wherein R¹⁵ is oxo.

Other preferred compounds of formula Ia and Ib are such wherein R¹⁵ is the group=NOH.

Other preferred compounds of formula Ia and Ib are such wherein R¹⁵ is a group of the general formula=NOR³², wherein R³² is C₁-C₃ alkyl.

Other preferred compounds of formula Ia and Ib are such wherein R¹⁵, together with R¹⁴, designates an additional bond between the carbon atoms at which R¹⁵ and R¹⁴ are placed.

Other preferred compounds of formula Ia and Ib are such wherein R¹⁶ is hydrogen.

Other preferred compounds of formula Ia and Ib are such wherein R¹⁶ is halogen.

Other preferred compounds of formula Ia and Ib are such wherein R¹⁶ is C₁-C₃ alkyl.

Other preferred compounds of formula Ia and Ib are such wherein R¹⁶ is methylene.

Other preferred compounds of formula Ia and Ib are such wherein R¹⁶ is methoxy.

Other preferred compounds of formula Ia and Ib are such wherein R¹⁶ is hydroxy.

Other preferred compounds of formula Ia and Ib are such wherein R¹⁶ is methoxy.

Other preferred compounds of formula Ia and Ib are such wherein R¹⁶ is the group=NOH.

Other preferred compounds of formula Ia and Ib are such wherein R¹⁶ is a group of the general formula=NOR³⁴, wherein R³⁴ is C₁-C₃ alkyl.

Other preferred compounds of formula Ia and Ib are such wherein R¹⁶ together with R¹⁷, designates an additional bond between the carbon atoms at which R¹⁶ and R¹⁷ are placed.

Other preferred compounds of formula Ia and Ib are such wherein R¹⁷ is hydrogen.

Other preferred compounds of formula Ia and Ib are such wherein R¹⁷ is hydroxy.

Other preferred compounds of formula Ia and Ib are such wherein R¹⁷ is in the a position.

Other preferred compounds of formula Ia and Ib are such wherein R²⁰ is hydrogen.

Other preferred compounds of formula Ia and Ib are such wherein R²⁰ is hydroxymethyl.

Other preferred compounds of formula Ia and Ib are such wherein R²⁰ is C₁-C₄ alkyl.

Other preferred compounds of formula Ia and Ib are such wherein R²⁰ together with R′²⁰ designates methylene.

Other preferred compounds of formula Ia and Ib are such wherein R²⁰ together with R′²⁰ designates oxo.

Other preferred compounds of formula Ia and Ib are such wherein R′²⁰ is hydrogen.

Other preferred compounds of formula Ia and Ib are such wherein R′²⁰ is halogen.

Other preferred compounds of formula Ia and Ib are such wherein R′²⁰ is methyl.

Other preferred compounds of formula Ia and Ib are such wherein R′²⁰ is hydroxy.

Other preferred compounds of formula Ia and Ib are such wherein R′²² is hydrogen.

Other preferred compounds of formula Ia and Ib are such wherein R²² is 3-methylbutyl.

Other preferred compounds of formula Ia and Ib are such wherein R²² is isobutyl.

Other preferred compounds of formula Ia and Ib are such wherein R²² is phenyl.

Other preferred compounds of formula Ia and Ib are such wherein the long side chain in the 17 position, i.e. —C(R²⁰) (R′²⁰)—CH(R′²²)—C(R²³) (R′²³)—C(R²⁴) (R′²⁴)—A(R²⁵)(R′²⁵) (R″²⁵), is in the a position.

It is to be understood that the above preferred substituents can be combined in any way with each other.

Examples of interesting and preferred compounds of the general formula Ia and Ib are as follows: Cholest-5-en-16β-ol; cholest-5-en-16-one; 4,4-dimethylcholesta-2,5-dien-16β-ol; cholestan-16β-ol; cholesta-3,5-dien-16β-ol; cholest-5-en-15β-ol; cholest-5-en-17α-ol; cholest-5-en-15α-ol; cholest-5-en-16α-ol; 4,4-dimethylcholest-5-en-16β-ol; cholest-3-en-16β-ol; cholest-4-en-16β-ol; cholest-2-en-16β-ol; cholesta-2,4-dien-16β-ol; cholesta-2,5-dien-16β-ol; cholesta-5,24-dien-16β-ol; cholesta-5,8-dien-16β-ol; cholesta-5,7-dien-16β-ol; 4,4-dimethylcholesta-5,7-dien-16β-ol; 3-methylcholesta-2,5-dien-16β-ol; 3β-methylcholest-5-en-16β-ol; 3α-methylcholest-5-en-16β-ol; 3,4,4-trimethylcholesta-2,5-dien-16β-ol; 4,4-dimethyl-cholesta-5,8-dien-16β-ol; cholesta-5,8-dien-15β-ol; cholesta-5,7-dien-15β-ol; 4,4-dimethyl-cholest-5-en-15β-ol; 4,4-dimethylcholest-5-en-15α-ol; 20-methyl-21-phenylpregna-5-en-16β-ol; 20-methyl-21-cyclopentylpregna-5-en-16β-ol; 24-norcholest-5-en-16β-ol; 24-norcholest-16β-ol; 24-norcholest-5-en-15β-ol; 20-methyl-21-(3-methylphenyl)pregna-5-en-16β-ol; 20-methyl-21-(3-hydroxyphenyl)pregna-16β-ol; 20-methyl-21 -(3-methylphenyl)pregna-15β-ol; 4,4,20-trimethyl-(4-methylphenyl)-pregna-5-en-16β-ol; 16β-hydroxychol-5-en-24-oic acid cyclohexyl ester; cholesta-5-en-16β,25-diol; 24-nor-cholestan-15β-ol; 20-methyl-21-benzylpregna-3,5-dien-16β-ol; 24-nor-4,4-dimethylcholest-5-en-16β-ol; 4,4,20-trimethyl-21 -(cyclopentyl)pregna-5-en-16β-ol; 16β-hydroxycholesta-5-en-24-one; (20S)-cholest-5-ene-16β,20-diol; (20R)-cholest-5-ene-16β,20-diol; (20S)-24-norcholest-5-ene-16P,20-diol; (20R)-24-norcholest-5-ene-16β,20-diol; (20S)-cholest-5,24-diene-16β,20-diol; (20R)-cholest-5,24-diene-16β,20-diol; (20S)-24-norcholest-5,23-diene-16β,20-diol; (20R)-24-norcholest-5,23-diene-16β,20-diol; (20S)-23,24-dinor-cholest-5-ene-16β,20-diol; (20R)-23,24-dinorcholest-5-ene-16β,20-diol; (20S)-20-methyl-21-phenylpregna-5-ene-16β,20-diol; (20R)-20-methyl-21 -phenylpregna-5-ene-16β,20-diol; (20S)-16β,20-dihydroxychol-5-en-24-oic acid-N-dimethyl amide; (20R)- 16β,20-dihydroxychol-5-en-24-oic acid-N-dimethyl amide; (20S)-20-hydroxychol-5-en-24-oic acid-N-dimethyl amide; (20R)-20-hydroxychol-5-en-24-oic acid-N-dimethyl amide; 16β-hydroxycholest-5-ene; cholest-5-ene-16-one; 16β-hydroxycholestane; and (25R)-16β,26-dihydroxycholest-5-ene.

Preferred compounds of formula Ia and Ib are such which when tested by the method described below for agonistic properties (penultimate example, below) shows a relative activity of at least 50, preferably at least 80, or when tested by the method described below for antagonistic properties (last example, below) shows a IC₅₀ value below 10 JIM, preferably below 2 μM.

Examples of other preferred compounds are such not being active at the estrogen receptor, and preferably compounds not being active at other presently known hormone receptors. Examples of such other hormone receptors are the progesterone receptor, the androgen receptor and the glucocorticoid receptor. Also, the compounds should not affect the entire oocyte reserve of ovaries.

Further preferred embodiments are mentioned in the appended claims.

As used in the present description and claims, a lower alkyl group - when used alone or in combinations—may be a straight or branched alkyl group. Preferably, said alkyl group contains not more than 6 carbon atoms. Preferred examples of lower alkyl groups are methyl, ethyl, propyl, isopropyl, butyl, tert-butyl, pentyl and hexyl, more preferred methyl, ethyl, propyl, isopropyl, butyl and tert-butyl, still more preferred methyl and ethyl. In a preferred embodiment of this invention, the lower alkyl group contains not more than 4 carbon atoms, preferably not more than 3 carbon atoms.

As used in the present description and claims, lower alkoxy designates a straight or branched alkoxy group preferably containing not more than 6 carbon atoms, preferably not more than 4, more preferred not more than 3 carbon atoms. Preferred examples are methoxy, ethoxy and propoxy, more preferred methoxy and ethoxy.

As used in the present description and claims, the expression halogen preferably designates fluoro and chloro, more preferred fluoro.

As used in the present description and claims, the expression C₃-C₆ cycloalkyl designates a cycloalkyl group containing 3-6 carbon atoms in the ring. Preferred examples are cyclopropyl and cyclopentyl.

As used in the present description and claims, the expression acyloxy designates a monovalent substituent comprising an optionally substituted C₁₋₆-alkyl or phenyl group linked through a carbonyloxy group; such as e.g. acetoxy, propionyloxy, butyryloxy, isobutyryloxy, pivaloyloxy, valeryloxy, benzoyl and the like.

As used in the present description and claims, a statement that, e.g., R¹ is oxo means that oxo (═O) is present in the 1 position (consequently, there is no hydrogen atom in the 1 position). Analogous considerations apply for similar situations. In other instances, two symbols together may represent oxo, e.g., R⁴ and R′⁴.

As used in the present description and claims, a statement that, e.g., R¹² is methylene means that methylene (═CH₂) is present in the 12 position and, consequently, there is no hydrogen atom in this position. Analogous considerations apply for similar situations. In other instances, two symbols together may represent methylene, e.g., R⁴ and R′⁴.

Salts of compounds of formula Ia and Ib are preferably pharmaceutically acceptable salts, especially acid-addition salts, including salts of organic acids and mineral acids. Examples of such salts include salts of organic acids such as formic acid, fumaric acid, acetic acid, propionic acid, glycolic acid, lactic acid, pyruvic acid, oxalic acid, succinic acid, malic acid, tartaric acid, citric acid, benzoic acid, salicylic acid and the like. Suitable inorganic acid-addition salts include salts of hydrochloric, hydrobromic, sulphuric and phosphoric acids and the like. Further examples of pharmaceutically acceptable inorganic or organic acid addition salts include the pharmaceutically acceptable salts listed in Journal of Pharmaceutical Science, 66 (1977), 2 et seq.

Esters of compounds of the general formula Ia or Ib are formally derived by esterification of one or more hydroxylic groups of a compound of formula Ia or Ib with an acid which can, for example, be selected from the group of acids comprising succinic acid and other aliphatic dicarboxylic acids, nicotinic acid, isonicotinic acid, ethylcarbonic acid, phosphoric acid, sulphonic acid, sulphamic acid, benzoic acid, acetic acid, propionic acid and other aliphatic monocarboxylic acids.

A metabolite of a compound of formula Ia or Ib is an active derivative of a compound of formula Ia or Ib which is produced when the compound of formula Ia or Ib is metabolized. Metabolites of compounds of formula Ia or Ib can be identified either by administration of a compound of formula Ia or Ib to a host and an analysis of blood samples from the host, or by incubation of a compound of formula Ia or Ib with hepatic cells in vitro and analysis of the incubant.

A prodrug of a compound of formula Ia or Ib is a compound that either is converted into a compound of formula Ia or Ib in vivo or which has the same active metabolites as a compound of formula Ia or lb.

The compounds of formula Ia and Ib have a number of chiral centers in the molecule and thus exists in several isomeric forms. All these isomeric forms and mixtures thereof are within the scope of the invention.

The compounds of formula Ia and Ib can be prepared analogously with the preparation of known compounds. Hence, synthesis of the compounds of formula Ia and Ib can followed the well established synthetic pathways described in the comprehensive sterol and steroid literature. The following books can be used as the key source in the synthesis: L. F. Fieser & M. Fieser: Steroids: Reinhold Publishing Corporation, NY 1959; Rood's Chemistry of Carbon Compounds (editor: S. Coffrey): Elsevier Publishing Company, 1971; J. Fried and J. A. Edwards: Organic Reactions in Steroid Chemistry, Vol. I and II, Van Nostrand Reinhold Company, New York, 1972; and especially Dictionary of Steriods (editors: R. A. Hill; D. N. Kirk; H. L. J. Makin and G. M. Murphy): Chapmann & Hall. The last one contains an extensive list of citations to the original papers covering the period up to 1990. All these books including the last mentioned citations are incorporated by reference. In addition, information in all the above publications (including patent specifications) dealing with preparation of compounds similar with compounds of formula Ia and Ib is incorporated by reference.

The compounds of the present invention will influence the meiosis in oocytes as well as in male germ cells.

The existence of a meiosis inducing substance in nature has been known for some time. However, until recently the identity of the meiosis inducing substance or substances was unknown.

The prospects of being able to influence the meiosis are several. According to a preferred embodiment of the present invention, a compound of formula Ia or Ib or an ester, salt, active metabolite and prodrug thereof can be used to stimulate the meiosis. According to another preferred embodiment of the present invention, a compound of formula Ia or Ib or an ester, salt, active metabolite and prodrug thereof can be used to stimulate the meiosis in humans. Thus, the compounds of formula Ia or Ib and esters, salts, active metabolites and prodrugs thereof are promising as new fertility regulating agents without the usual side effect on the somatic cells which are known from the hitherto used hormonal contraceptives which are based on estrogens and/or gestagens.

For use as a contraceptive agent in females, a meiosis inducing substance can be administered so as to prematurely induce resumption of meiosis in oocytes while they are still in the growing follicle, before the ovulatory peak of gonadotropins occurs. In women, the resumption of the meiosis can, for example, be induced a week after the preceding menstruation has ceased. When ovulated, the resulting overmature oocytes are then most likely not to be fertilized. The normal menstrual cycle is not likely to be affected. In this connection it is important to notice, that the biosynthesis of progesterone in cultured human granulosa cells (somatic cells of the follicle) is not affected by the presence of a meiosis inducing substance whereas the estrogens and gestagens used in the hitherto used hormonal contraceptives do have an adverse effect on the biosynthesis of progesterone.

According to another aspect of this invention, a meiosis inducing substance of formula la or Ib or an ester, salt, active metabolite and prodrug thereof can be used in the treatment of certain cases of infertility in females, including women, by administration thereof to females who, due to an insufficient own production of meiosis activating substance, are unable to produce mature oocytes. Also, when in vitro fertilization is performed, better results can be achieved, when a compound of formula Ia or Ib or an ester, salt, active metabolite and prodrug thereof is added to the medium in which the oocytes are cultured.

When infertility in males, including men, is caused by an insufficient own production of the meiosis activating substance and thus a lack of mature sperm cells, administration of a compound of formula Ia or Ib or an ester, salt, active metabolite and prodrug thereof may relieve the problem.

As an alternative to the method described above, contraception in females can also be achieved by administration of a compound of formula Ia or Ib or an ester, salt, active metabolite and prodrug thereof which inhibits the meiosis, so that no mature oocytes are produced. Similarly, contraception in males can be achieved by administration of a compound of formula Ia or Ib or an ester, salt, active metabolite and prodrug thereof which inhibits the meiosis, so that no mature sperm cells are produced.

The route of administration of compositions containing a compound of formula Ia or Ib or an ester, salt, active metabolite and prodrug thereof may be any route which effectively transports the active compound to its site of action.

Thus, when the compounds of formula Ia or Ib are to be administered to a mammal, they are conveniently provided in the form of a pharmaceutical composition which comprises at least one compound of formula Ia or Ib or an ester, salt, active metabolite and prodrug thereof in connection with a pharmaceutically acceptable carrier. For oral use, such compositions are preferably in the form of capsules or tablets.

From the above it will be understood that administrative regimen called for will depend on the condition to be treated. Thus, when used in the treatment of infertility the administration may have to take place once only, or for a limited period, e.g. until pregnancy is achieved. When used as a contraceptive, the compounds of formula Ia or Ib or esters, salts, active metabolites and prodrugs thereof will either have to be administered continuously or cyclically. When used as a contraceptive by females and not taken continuously, the timing of the administration relative to the ovulation will be important.

Pharmaceutical Compositions

Pharmaceutical compositions comprising a compound of formula Ia or Ib or an ester, salt, active metabolite and prodrug thereof may further comprise carriers, diluents, absorption enhancers, preservatives, buffers, agents for adjusting the osmotic pressure, tablet disintegrating agents and other ingredients which are conventionally used in the art. Examples of solid carriers are magnesium carbonate, magnesium stearate, dextrin, lactose, sugar, talc, gelatin, pectin, tragacanth, methyl cellulose, sodium carboxymethyl cellulose, low melting waxes and cocoa butter.

Liquid compositions include sterile solutions, suspensions and emulsions. Such liquid compositions may be suitable for injection or for use in connection with ex vivo and in vitro fertilization. The liquid compositions may contain other ingredients which are conventionally used in the art, some of which are mentioned in the list above.

Further, a composition for transdermal administration of a compound of this invention may be provided in the form of a patch and a composition for nasal administration may be provided in the form of a nasal spray in liquid or powder form.

The dose of a compound of a compound of formula Ia or Ib to be used will be determined by a physician and will depend, inter alla, on the particular compound employed, on the route of administration and on the purpose of the use. In general, the compositions of the invention are prepared by intimately bringing into association the active compound with the liquid or solid auxiliary ingredients and then, if necessary, shaping the product into the desired formulation.

Usually, not more than 1000 mg, preferably not more than 100 mg, and in some preferred instances not more than 10 mg, of a compound of formula Ia or Ib is to be administered to mammals, e.g. to man, per day.

None of the compounds of formula Ia and Ib have been shown to be toxic when administered to man in an amount of 1000 mg per day.

The present invention is further illustrated by the following examples which, however, are not to be construed as limiting the scope of protection. The features disclosed in the foregoing description and in the following examples may, in any combination thereof, be material for realizing the invention in diverse forms thereof.

### EXAMPLE 1

16β-Hydroxycholest-5-ene

To a mixture of (25R)-cholest-5-ene-3β,16β,26-triol (10 g, 24 mmol; prepared according to the procedure described by Arunachalam et al. in J. Org. Chem. 46 (1981), 2966-2968), pyridine (150 mL) and dichloromethane (150 mL) was added toluene sulphonyl chloride (5.7 g, 30 mmol) and the mixture stirred overnight. Ice water was added and the aqueous phase extracted with dichloromethane. The organic phase was washed with 4N HCl, concentrated under reduced pressure and the residue was purified by flash chromatography to give (25R)-3,26-ditosylcholest-5-ene-16β-ol (4.2 g) and (25R)-26-tosyloxycholest-5-ene-3,16-diol (6.3 g). The ¹H-NMR spectrum (CDCl₃, δ) showed characteristic signals at: 2.43 (s, 6H), 3.45 (q, 1 H), 3.72-3.92 (m, 2H), 4.25-4.40 (m, 1H), 5.23 (m, 1 H), 7.32 (m, 4H), 7.78 (m, 4H).

To a solution of (25R)-3,26-ditosylcholest-5-ene-16β-ol (360 mg, 0.5 mmol) in tetrahydrofuran (hereinafter designated THF; 5 mL) was added 1 M lithium triethylborohydride (16 mL). Water was added and the aqueous phase extracted with dichloromethane and washed with dilute HCl, aqueous sodium bicarbonate, and brine. Concentration under reduced pressure and purification by flash chromatography gave the title compound (60 mg), melting point 107-108° C. The ¹H-NMR spectrum (CDCl₃, δ) showed characteristic signals at: 0.83 (s, 3H), 0.90 (s, 3H), 1.00 (s, 3H), 2.15-2.3 (m, 2H), 4.30-4.41 (m, 1H, H-16), 5.25 (d, 1H. H-6). The ¹³C-NMR spectrum (CDCl₃, δ) showed characteristic signals at: 72.9 (C-16), 119.1 (C-6), 144.2 (C-5). The mass spectrum showed characteristic peaks at: 386.4 (M⁺).

### EXAMPLE 2

Cholest-5-ene-16-one

16β-Hydroxycholest-5-ene (example 1, 80 mg, 0.2 mmol) was dissolved in glacial acetic acid (4 mL) and sodium acetate trihydrate (680 mg, 5 mmol) was added followed by dropwise addition of chromium trioxide (20 mg, 0.2 mmol) in glacial acetic acid and water (0.3 mL of a 2:1 mixture). After 2 hours, methanol (2 mL) was added and the mixture concentrated. Water was added and the aqueous phase extracted with dichloromethane. Combined organic layers were washed with sodium bicarbonate, water and brine. Removal of solvent and recrystallisation from methanol gave the title compound (18 mg). The ¹H-NMR spectrum (CDCl₃, δ) showed characteristic signals at 5.25 (d, 1 H. H-6). The ¹³C-NMR spectrum (CDCl₃, δ) showed characteristic signals at: 118.6 (C-6)144.2 (C-S), 219.3 (C-16). The mass spectrum showed characteristic peaks at: 384.2 (M⁺).

### EXAMPLE 3

16β-Hydroxycholestane

16β-Hydroxycholest-5-ene (example 1, 20 mg) was hydrogenated under atmospheric pressure over platinum on charcoal. Filtration and chromatography gave the title compound (17 mg). The ¹H-NMR spectrum (CDCl₃, δ) showed characteristic signals at 4.30-4.40 (m, 1 H. H-16). The mass spectrum showed characteristic peaks at: 388.3 (M⁺).

### EXAMPLE 4

(25R)-160.26-Dihydroxycholest-5-ene

A solution of tetrahydrodiosgenin (2.5 g, 5.9 mmol), tert-butyidimethylsilylchloride (1.1 g, 7.1 mmol) and imidazole (1.6 g, 24 mmol) in dimethylformamide was stirred for 48 hours at 40° C., poured into water (200 mL) and extracted with ethyl acetate. Purification by flash chromatography gave (25R)-3,16β-dihydroxy-26-tert-butyidimethylsilyloxycholest-5-ene (1.3 g). The ¹H-NMR spectrum (CDCl₃, δ) showed characteristic signals at: −0.05-0.03 (d, 6H), 0.88 (s, 9H), 3.30-3.60 (3H, m, H-3 and 2H-26), 4.30-4.40(m, 1 H, H-16), 5.35 (m, 1 H. H-6).

(25R)-3,16β-Dihydroxy-26-tert-butyldimethylsilyloxycholest-5-ene (0.76 g, 1.4 mmol) and toluene sulphonylchloride (0.54 g, 2.8 mmol) in pyridine (20 mL) was stirred for 48 hours at room temperature. Concentration under reduced pressure and flash chromatography afforded (25R)-3-tosyloxy-16, phydroxy-26-tert-butyidimethylsilytoxycholest-5-ene (0.855 g). The ¹H-NMR spectrum (CDCl₃, δ) showed characteristic signals at: -0.05-0.03 (d, 6H), 0.88 (s, 9H), 2.45 (s, 3H), 3.30-3.49 (2H, m, 2H-26), 4.30-4.40 (m, 2H, H-3 and H-16), 5.30 (m, 1H. H-6), 7.30 (d, 2H), 7.73 (d, 2H).

To (25R)-3-tosyloxy-16β-hydroxy-26-tert-butyidimethylsilyloxycholest-5-ene (0.85 g, 1.2 mmol) was added Super Hydride (30 mL of 1 M in THF) and the reaction stirred for 72 hours at room temperature, poured into ice water and extracted with ethyl acetate. Removal of solvent under reduced pressure and flash chromatography gave 16β-hydroxy-26-tertbutyldimethylsilyloxycholest-5-ene (0.53 g). The ¹H-NMR spectrum (CDCl₃, δ) showed characteristic signals at: -0.05-0.03 (d, 6H), 0.88 (s, 9H), 3.30-3.49 (2H, m, 2H-26), 4.30-4.40 (m, 1H, H-16), 5.30 (m, 1H. H-6).

To 16β-hydroxy-26-tert-butyidimethylsilyloxycholest-5-ene in THF was added tetrabutyl ammoniumfluoride (0.6 g) and the reaction stirred overnight at room temperature. Removal of solvent under reduced pressure and flash chromatography gave the title compound. The ¹H-NMR spectrum (CDCl₃, δ) showed characteristic signals at: 3.40-3.52 (2H, m, 2H-26), 4.30-4.40 (m, 1H, H-16), 5.30 (d, 1H. H-6).

### EXAMPLE 5

An agonistic oocyte assay can be performed as follows:

Oocytes were obtained from immature female mice (C57BL/6J×DBA/2J F1, Bomholtgaard, Denmark) weighing 13-16 grams, that were kept under controlled temperature (20-22° C.), light (lights on 06.00-18.00) and relative humidity (50-70%). The mice received an intra-peritoneal injection of 0.2 ml gonadotropins (Gonal-F, Serono) containing 20 IU FSH and 48 hours later the animals were killed by cervical dislocation.

The ovaries were dissected out and the oocytes were isolated in Hx-medium (see below) under a stereo microscope by manual rupture of the follicles using a pair of 27 gauge needles. Spherical oocytes displaying an intact germinal vesicle (hereinafter designated GV) were divided in cumulus enclosed oocytes (hereinafter designated CEO) and naked oocytes (hereinafter designated NO) and placed in a-minimum essential medium (α-MEM without ribonucleosides, Gibco BRL, Cat. No. 22561) supplemented with 3 mg/ml bovine serum albumin (BSA, Sigma Cat. No. A-7030), 5 mg/ml human serum albumin (HSA, Statens Seruminstitute, Denmark), 0.23 mM pyruvate (Sigma, Cat. No S-8636), 2 mM glutamine (Flow Cat. No. 16-801), 100 IU/ml penicillin and 100 μg/ml streptomycin (Flow, Cat No. 16-700). This medium was supplemented with 3 mM hypoxanthine (Sigma Cat. No. H-9377) and designated Hx-medium.

The oocytes were rinsed three times in Hx-medium and oocytes of uniform size were divided into groups of CEO and NO. CEO and NO were cultured in 4-well multidishes (Nunclon, Denmark) in which each well contained 0.4 ml of Hx-medium. One control well (i.e., 35-45 oocytes cultured in identical medium with no addition of test compound) was always cultured simultaneously with 3 test wells (35-45 oocytes per well supplemented with test compound).

The oocytes were cultured in a humidified atmosphere of 5% CO₂ in air for 24 hours at 37° C. By the end of the culture period, the number of oocytes with germinal vesicle (hereinafter designated GV), germinal vesicle breakdown (hereinafter designated GVB) and polar bodies (hereinafter designated PB), respectively, were counted using a stereomicroscope (Wildt, Leica MZ 12). The percentage GVB, defined as percentage of oocytes undergoing GVB per total number of oocytes in that well, was calculated as:

% GVB=(number of GVB+number of PB/total number oocytes)×100.

The % PB was defined as percentage of oocytes displaying one extruded polar body per total number of oocytes in that well.

The effect of the tested compounds has been indexed against control level and 4,4-5 dimethyl-5β-cholesta-8,14,24-trien-39-ol (hereinafter designated FF-MAS) where controls and FF-MAS are indexed to an effect of 0 and 100, respectively. The relative effect of the tested compound is calculated as follows:

Relative effect=((test GVB %−control GVB %)/(FF-MAS GVB %−control GVB %))×100.

Using this assay on the compounds prepared in Examples 1 and 4, a GVB of 72 and 63%, respectively, was found and the relative GVB was 88 and 73%, respectively.

### EXAMPLE 6

An antagonistic oocyte assay can be performed as follows:

Animals

20 Oocytes were obtained from immature female mice (C57BI/6J×DBA/2J F1-hybrids, Bomholtgaard, Denmark) weighing 13-16 grams, that were kept under controlled lighting and temperature. The mice received an intra-peritoneal injection of 0.2 ml gonadotropins (Gonal F, Serono, Solna, Sweden, containing 20 IU FSH, alternatively, Puregon, Organon, Swords, Ireland containing 20 IU FSH) and 48 hours later the animals were killed by cervical dislocation.

Test of Meiosis-Inhibiting Substances in the Oocyte Test

The ovaries were dissected out and the oocytes were isolated in Hx-medium (see below) under a stereo microscope by manual rupture of the follicles using a pair of 27 gauge needles. Spherical, naked oocytes (NO) displaying an intact germinal vesicle (GV) were placed in α-minimum essential medium (α-MEM without ribonucleosides, Gibco BRL, Cat.No. 22561) supplemented with 3 mM hypoxanthine (Sigma Cat. No. H-9377), 8 mg/ml human serum albumin (HSA, Statens Seruminstitut, Denmark), 0.23 mM pyrubate (Sigma, Cat. No. S-8636), 2 mM glutamine (Flow Cat. No. 16-801), 100 IU/ml penicillin and 100 μg/ml streptomycin (Flow, Cat No. 16-700). This medium was designated Hx-medium.

Naked oocytes (NO) were rinsed three times in Hx-medium. 4,4-Dimethyl-5α-cholesta-8,14,24-trien-3β-ol (FF-MAS) has previously been shown to induce meiosis in NO in vitro (Byskov, A. G. et al. Nature 374 (1995), 559-562). NO were cultured in Hx-medium supplemented with 5 μM FF-MAS in co-culture with the test compounds in different concentrations in 4-well multidishes (Nunclon, Denmark) in which each well contained 0.4 ml of the medium and 35-45 oocytes. One positive control (i.e., 35-45 oocytes cultured in Hx-medium containing FF-MAS with no addition of test compound) was always run simultaneously with the test cultures, which were supplemented with different concentrations of the compounds to be tested. In addition, one negative control (35-45 oocytes cultured in Hx-medium alone) was run simultaneously with the positive control.

Examination of Oocytes

By the end of the culture period, the number of oocytes with germinal vesicle (GV) or germinal vesicle breakdown (GVB) and those with polar body (PB) was counted using a stereo-microscope or an inverted microscope with differential interference contrast equipment. The percentage of oocytes with GVB+PB per total number of oocytes were calculated in the test cultures and in the control (positive and negative) culture groups. The relative inhibition of the test compound was calculated by the following formula:

Inhibition of test compound (in percentage)=100−[(GVB_{test Compound}−GVB_{negative control})×100/(GVB_{positive control}−GVB_{negative control})].

In case of a dose response curve, an IC₅₀ (dose, which lead to a 50% inhibition) was calculated.

Using this assay on the compound prepared in Example 4, a PB of 5% was found.

### EXAMPLE 7

An in vitro fertilization (IVF) assay can be performed as follows:

Naked oocytes (NO) and cumulus enclosed oocytes (CEO) from immature mice (C57B1 16J×DBAJ/2)F, were isolated and cultured under the same conditions as described for the agonistic oocyte assay (Example 5). After 18 hours oocytes that exhibited germinal vesicle breakdown (GVB) were shortly washed in hypoxanthine-free medium and transferred to the insemination dishes prepared in advance, which consisted of a motile sperm preparation from the caudal epididymis of male mice. The dishes were then incubated under defined gas conditions (5% CO₂) at 37° C. in a modified (α-MEM IVF-medium. Neither the insemination medium nor the IVF-medium contained hypoxanthine. Examination of the oocytes was carried out 20-22 hours after insemination, in order to check fertilization and to record the number of 2-cell embryos. The percentage fertilization (=fertilization rate) was determined from counts of oocytes that had cleaved into two-cell embryos.

Using this assay on the compound prepared in Example 1, a fertilization rate of 62% was found (the fertilization rate in control animals was 22%).

What is claimed is:

1. A compound of formula Ia: 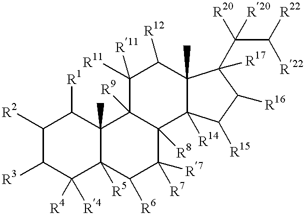
wherein R¹ is hydrogen, halogen, methyl, hydroxy or oxo; R² is selected from the group consisting of hydrogen, hydroxy, lower alkyl, vinyl, lower alkoxy and halogen, or R² designates, together with R³, an additional bond between the carbon atoms at which R² and R³ are placed; R³ is hydrogen or lower alkyl; or R³ designates, together with R⁴, an additional bond between the carbon atoms at which R³ and R⁴ are placed; or R³ designates, together with R², an additional bond between the carbon atoms at which R² and R³ are placed; R⁴ and R′⁴, which are different or identical provided that they are not both hydroxy, are selected from the group consisting of hydrogen, halogen, hydroxy and lower alkyl which may be substituted by halogen, hydroxy or cyano, or wherein R⁴ and R′⁴ together designate methylene or oxo or, together with the carbon atom to which they are bound, form a cyclopropane ring, a cyclopentane ring, or a cyclohexane ring; or R⁴, R′⁴ and R⁵ together designate an additional bond between the carbon atoms in the 4 and 5 position, R⁵ is hydrogen, halogen or hydroxy, or R⁵ designates, together with R⁶, an additional bond between the carbon atoms at which R⁵ and R⁶ are placed; R⁵ is hydrogen, hydroxy, halogen or oxo, or R⁶ designates, together with R⁵ or R⁷, an additional bond between the carbon atoms at which R⁶ and R⁵ or R⁷ are placed; R⁷ is selected from the group consisting of hydrogen, hydroxy, methoxy, acyloxy, halogen and lower alkyl, or R⁷ designates, together with R⁶ or R⁸, an additional bond between the carbon atoms at which R⁷ and R⁶ or R⁸ are placed; and R′⁷ is hydrogen or, if R⁷ is lower alkyl, R′⁷ is hydrogen or hydroxy; or R⁷ and R′⁷ together designates oxo, methylene or a group of the formula=NOR³⁶ wherein R³⁶ is hydrogen or lower alkyl; R⁸ is hydrogen, hydroxy or halogen, or R⁸ designates, together with R⁷, R⁹ or R¹⁴, an additional bond between the carbon atoms at which R⁸ and R⁷, R⁹ or R¹⁴ are placed; R⁹ is hydrogen, hydroxy or halogen, or R⁹ designates, together with R⁸ or R¹¹, an additional bond between the carbon atoms at which R⁹ and R⁸ or R¹¹ are placed; R¹¹ is selected from the group consisting of hydrogen, hydroxy, methoxy, acyloxy, halogen and lower alkyl, or R¹¹ designates, together with R⁹ or R¹², an additional bond between the carbon atoms at which R¹¹ and R⁹ or R¹² are placed; and R′¹¹ is hydrogen or, if R¹¹ is lower alkyl, R′¹¹ is hydrogen or hydroxy; or R¹¹ together with R′¹¹ is oxo, methylene or a group of the formula=NOR³⁷ wherein R³⁷ is hydrogen or lower alkyl; R¹² is selected from the group consisting of hydrogen, halogen, lower alkyl, methylene, hydroxy, lower alkoxy, acyloxy, oxo and a group of the formula=NOR³³ wherein R³³ is hydrogen or lower alkyl, or R¹² designates, together with R¹¹, an additional bond between the carbon atoms at which R¹¹ and R¹² are placed; R¹⁴ is hydrogen or hydroxy, or R¹⁴ designates, together with R¹⁵, an additional bond between the carbon atoms at which R¹⁴ and R¹⁵ are placed; R¹⁵ is selected from the group consisting of hydrogen, halogen, lower alkyl, methylene, hydroxy, lower alkoxy, oxo and a group of the formula=NOR³² wherein R³² is hydrogen or lower alkyl, or R¹⁵ designates, together with R¹⁴ an additional bond between the carbon atoms at which R¹⁵ and R¹⁴ are placed; R¹⁶ is selected from the group consisting of hydrogen, halogen, lower alkyl, methylene, hydroxy, lower alkoxy, oxo and a group of the general formula=NOR³⁴ wherein R³⁴ is hydrogen or lower alkyl, or R¹⁶ designates, together with R¹⁷, an additional bond between the carbon atoms at which R¹⁶ and R¹⁷ are placed; R¹⁷ is hydrogen or hydroxy, or R¹⁷ designates, together with R¹⁶, an additional bond between the carbon atoms at which R¹⁷ and R¹⁶ are placed; R²⁰ is selected from the group consisting of hydrogen, lower alkyl and hydroxymethyl, or R²⁰ and R′²⁰ together designate methylene or oxo; R′²⁰ is hydrogen, halogen, lower alkyl or hydroxy, R′²² is hydrogen, hydroxy or oxo; R²² represents a group of the formula:
—C(R²³) (R′²³)—C(R²⁴) (R′²⁴)—A(R²⁵) (R′²⁵) (R″²⁵),
wherein R²³ and R′²³ are both hydrogen, or one of R²³ and R′²³ is hydrogen while the other is halogen, hydroxy or methoxy, or R²³ and R′²³ together designates oxo, R′²⁴ is hydrogen when R²⁴ is different from oxo, and R′²⁴ is absent when R²⁴ is oxo; and A is a carbon atom or a nitrogen atom; and when A is a carbon atom, R²⁵ is selected from the group consisting of hydrogen, hydroxy and halogen; and R²⁴ is selected from the group consisting of hydrogen, halogen, hydroxy, lower alkyl, methylene and oxo, or R²⁵ designates, together with R²⁴, an additional bond between the carbon atoms at which R²⁴ and R²⁵ are placed; R′²⁵ is selected from the group consisting of lower alkyl, trifluoromethyl and C₃-C₆ cycloalkyl; R″²⁵ is selected from the group consisting of lower alkyl, hydroxy(lower alkyl), halogen(lower alkyl) containing up to three halogen atoms, methoxymethyl, acetoxymethyl, and C₃-C₆ cycloalkyl, or R′²⁵ and R″²⁵, together with the carbon atom at which they are placed, form a C₃-C₆ cycloalkyl ring; and when A is a nitrogen atom, R²⁵ designates a lone pair of electrons; and R²⁴ is selected from the group consisting of hydrogen, hydroxy, lower alkyl, cyano and oxo; and R′²⁵ and R″²⁵ are, independently, lower alkyl or C₃-C₆ cycloalkyl; or R²² is phenyl, toluyl, hydroxyphenyl, cyclopentyl, cyclohexyl, isobutyl or cyclohexyloxycarbonylmethyl; and esters, salts, active metabolites and prodrugs thereof, provided that the following compounds are disclaimed: cholest-4,8,24-triene; 25-aza-cholest-5-ene; cholest-3,5-diene; cholest-2-ene; cholest-5-ene; 24-methylcholesta-3,5-diene; 24-ethylcholesta-3,5,22-diene; 24-ethylcholest-3,5-diene; 24-nor-5α-cholestane; 24-nor-5β-cholestane; (20R)-5β,14α,17α(H)-cholestane; (20R)-5α,14β,17β(H)-cholestane; (20R)-5α,14α,17α(H)-cholestane; (20R)-5β,14α,17α(H)-24-methylcholestane; (20R)-5α,14α,17α(H)-24-ethylcholestane; (20R)-5α,14α,17α(H)-24-ethylcholestane; 4-methyl-5α-24-norcholestane; 5α-cholestane; 5β-cholestane; cholest-4-ene; 4-methylcholestane; (20S)-5α,14α,17α-cholestane; 19-nor-5α-cholestane; cholest-4-ene; norcholestane; 5α,8β,14β-cholestane; 5β,8β,14β-cholestane; 5α-norcholestane; 5α,17β(H)-cholestane; (20S)-5α,17β(H)-cholestane; (24R)-24-methyl-5β-cholestane; (24S)-24-methyl-5β-cholestane; 5α,8α,14β-cholestane; (20S)-5α-cholestane; (24R)-24-methyl-5α-cholestane; (24S)-24-ethyl-5α-cholestane; (24S)-24-ethyl-5α-cholestane; 4α-methyl-5α-cholestane; 4β-methyl-5α-cholestane; (24S)-24-methyl-5α-cholestane; 24-hydroxymethyl-chola-24-one; 24-hydroxymethylchola-24-ol; cholest-24-ene; 24-cyclohexylchola-24-ene; 24-methylcholest-2-ene; 24-ethylcholest-2-ene; 24-propylcholestane; cholest-25-ene; 24-cyclo-hexylchola-24-ene; and 24-dimethyl-5α-cholane.

2. A compound according to claim 1, wherein R¹ is hydrogen; R² is selected from the group consisting of hydrogen, or R² desinates, together with R³, an additional bond between the carbon atoms at which R² and R³ are placed; R³ is hydrogen or lower alkyl; or R³ designates, together with R⁴, an additional bond between the carbon atoms at which R³ and R⁴ are placed; R⁴ and R′⁴, which are different or identical provided that they are not both hydroxy, are selected from the group consisting of hydrogen, hydroxy and lower alkyl, or wherein R⁴, R′⁴ and R⁵ together designate an additional bond; R⁵ is hydrogen, or R⁵ designates, together with R⁶, an additional bond between the carbon atoms at which R⁵ and R⁶ are placed; R⁶ is hydrogen, or R⁶ designates, together with R⁵ or R⁷, an additional bond between the carbon atoms at which R⁶ and R⁵ or R⁷ are placed; R⁷ is hydrogen or hydroxy, or R⁷ designates, together with R⁶ or R⁸, an additional bond between the carbon atoms at which R⁷ and R⁶ or R⁸ are placed, and R′⁷ is hydrogen, or R⁷ and R′⁷ together designates oxo or methylene; R⁸ is hydrogen, or R⁸ designates, together with R⁷, R⁹ or R¹⁴, an additional bond between the carbon atoms at which R⁸ and R⁷, R⁹or R¹⁴ are placed; R⁹ is hydrogen, or R⁹ designates, together with R⁸ or R¹¹, an additional bond between the carbon atoms at which R⁹ and R⁸ or R¹¹ are placed; R¹¹ is hydrogen or hydroxy, or R¹¹ designates, together with R⁹, an additional bond between the carbon atoms at which R⁹ and R¹¹ are placed, and R′¹¹ is hydrogen; R¹² is hydrogen; R¹⁴ is hydrogen, or R¹⁴ designates, together with R¹⁵, an additional bond between the carbon atoms at which R¹⁴ and R¹⁵ are placed; R¹⁵ is hydrogen, hydroxy or oxo; R¹⁶ is hydrogen, hydroxy or oxo; or R¹⁶ designates, together with R¹⁷, an additional bond between the carbon atoms at which R¹⁶ and R¹⁷ are placed; R¹⁷ is hydrogen or hydroxy, or R¹⁷ designates, together with R′⁶, an additional bond between the carbon atoms at which R′⁷ and R¹⁶ are placed; R²⁰ is hydrogen or lower alkyl, or R²⁰ and R′²⁰ together designate methylene or oxo; R′²⁰ is hydrogen, halogen, lower alkyl or hydroxy, R′²² is hydrogen, hydroxy or oxo; and R²² is phenyl, toluyl, hydroxyphenyl, cyclopentyl, cyclohexyl, isobutyl, 3-methylbutyl or cyclohexyloxycarbonylmethyl; and esters, salts, active metabolites and prodrugs thereof.

3. A compound according to claim 1, which is cholest-5-en-16β-ol; cholest-5-en-16-one; 4,4-dimethylcholesta-2,5-dien-16β-ol; cholestan-16β-ol; cholesta-3,5-dien-16βol; cholest-5-en-15β-ol; cholest-5-en-17α-ol; cholest-5-en-15α-ol; cholest-5-en-16α-ol; 4,4-dimethylcholest-5-en-16β-ol; cholest-3-en-16β-ol; cholest-4-en-16β-ol; cholest-2-en-16β-ol; cholesta-2,4-dien-16β-cholesta-2,5-dien-16β-ol; cholesta-5,24-dien-16β-ol; cholesta-5,8-dien-16β-ol; cholesta-5,7-dien-16β-ol; 4,4-dimethylcholesta-5,7-dien-16β-ol; 3-methylcholesta-2,5-dien-16β-ol; 3β-methylcholest-5-en-16β-ol; 3α-methylcholest-5-en-16β-ol; 3,4,4-trimethylcholesta-2,5-dien-16β-ol; 4,4-dimethylcholesta-5,8-dien-16β-ol; cholesta-5,8-dien-15β-ol; cholesta-5,7-dien-15β-ol; 4,4-dimethylcholest-5-en-15β-ol; 4,4-dimethylcholest-5-en-15α-ol; 20-methyl-21-phenylpregna-5-en-16β-ol; 20-methyl-21-cyclopentylpregna-5-en-16β-ol; 24-norcholest-5-en-16β-ol; 24-norcholest-16βol; 24-norcholest-5-en-15β-ol; 20-methyl-21-(3-methylphenyl)pregna-5-en-16β-ol; 20-methyl-21-(3-hydroxyphenyl)pregna-5-en-16β-ol; 20-methyl-21 -(3-hydroxyphenyl)pregna-16β-ol; 20-methyl-21 -(3-methylphenyl)pregna-15β-ol; 4,4,20-trimethyl-(4-methylphenyl)pregna-5-en-16β-ol; 16 hydroxychol-5-en-24-oic acid cyclohexyl ester; cholesta-5-en-16β,25-diol; 24-nor-cholestan-15β-ol; 20-methyl-21-benzylpregna-3,5-dien-16βol; 24-nor-4,4-dimethylcholest-5-en-16β-ol; 4,4,20-trimethyl-21-(cyclopentyl)pregna-5-en-16β-ol; 16β-hydroxycholesta-5-en-24-one; (20S)-cholest-5-ene-16β,20-diol; (20R)-cholest-5-ene-16β,20-diol; (20S)-24-norcholest-5-ene-16β,20-diol; (20R)-24-norcholest-5-ene-16β,20-diol; (20S)-cholest-5,24-diene-16β,20-diol; (20R)-cholest-5,24-diene-16β,20-diol; (20S)-24-norcholest-5,23-diene-16β,20-diol; (20R)-24-norcholest-5,23-diene-16β,20-diol; (20S)-23,24-dinorcholest-5-ene-16β,20-diol; (20R)-23,24-dinorcholest-5-ene-16β,20-diol; (20S)-20-methyl-21-phenylpregna-5-ene-16β,20-diol; (20R)-20-methyl-21-phenylpregna-5-ene-16β,20-diol; (20S)-16β,20-dihydroxychol-5-en-24-oic acid-N-dimethyl amide; (20R)-16β,20-dihydroxychol-5-en-24-oic acid-N-dimethyl amide; (20S)-20-hydroxy-chol-5-en-24-oic acid-N-dimethyl amide; (20R)-20-hydroxychol-5-en-24-oic acid-N-dimethyl amide; 16β-hydroxycholest-5-ene; cholest-5-ene-16-one; 16β-hydroxycholestane; or (25R)-16β,26-dihydroxycholest-5-ene.

4. A medicament comprising a compound of formula Ib: 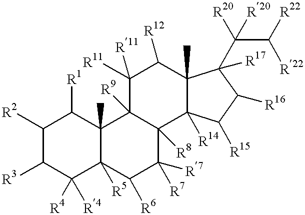
wherein R¹ is hydrogen, halogen, methyl, hydroxy or oxo; R² is selected from the group consisting of hydrogen, hydroxy, lower alkyl, vinyl, lower alkoxy and halogen, or R² designates, together with R³, an additional bond between the carbon atoms at which R² and R³ are placed; R³ is hydrogen or lower alkyl; or R³ designates, together with R⁴, an additional bond between the carbon atoms at which R³ and R⁴ are placed; or R³ designates, together with R², an additional bond between the carbon atoms at which R² and R³ are placed; R⁴ and R′⁴, which are different or identical provided that they are not both hydroxy, are selected from the group consisting of hydrogen, halogen, hydroxy and lower alkyl which may be substituted by halogen, hydroxy or cyano, or wherein R⁴ and R′⁴ together designate methylene or oxo or, together with the carbon atom to which they are bound, form a cyclopropane ring, a cyclopentane ring, or a cyclohexane ring; or R⁴, R′⁴ and R⁵ together designate an additional bond between the carbon atoms in the 4 and 5 position, R⁵ is hydrogen, halogen or hydroxy, or R⁵ designates, together with R⁶, an additional bond between the carbon atoms at which R⁵ and R⁶ are placed; R⁶ is hydrogen, hydroxy, halogen or oxo, or R⁶ designates, together with R⁵ or R⁷, an additional bond between the carbon atoms at which R⁶ and R⁵ or R⁷ are placed; R⁷ is selected from the group consisting of hydrogen, hydroxy, methoxy, acyloxy, halogen and lower alkyl, or R⁷ designates, together with R⁶ or R⁸, an additional bond between the carbon atoms at which R⁷ and R⁶ or R⁸ are placed; and R′⁷ is hydrogen or, if R⁷ is lower alkyl, R′⁷ is hydrogen or hydroxy; or R⁷ and R′⁷ together designates oxo, methylene or a group of the formula=NOR³⁶ wherein R³⁶ is hydrogen or lower alkyl; R⁸ is hydrogen, hydroxy or halogen, or R⁸ designates, together with R⁷, R⁹ or R¹⁴, an additional bond between the carbon atoms at which R⁸ and R⁷, R⁹or R¹⁴ are placed; R⁹ is hydrogen, hydroxy or halogen, or R⁹ designates, together with R⁸ or R¹¹, an additional bond between the carbon atoms at which R⁹ and R⁸ or R¹¹ are placed; R¹¹ is selected from the group consisting of hydrogen, hydroxy, methoxy, acyloxy, halogen and lower alkyl, or R¹¹ designates, together with R⁹ or R¹², an additional bond between the carbon atoms at which R¹¹ and R⁹ or R¹² are placed; and R¹¹ is hydrogen or, if R¹¹ is lower alkyl, R′¹¹ is hydrogen or hydroxy; or R¹¹ together with R′¹¹ is oxo, methylene or a group of the formula=NOR³⁷ wherein R³⁷ is hydrogen or lower alkyl; R¹² is selected from the group consisting of hydrogen, halogen, lower alkyl, methylene, hydroxy, lower alkoxy, acyloxy, oxo and a group of the formula=NOR³³ wherein R³³ is hydrogen or lower alkyl, or R¹² designates, together with R¹¹, an additional bond between the carbon atoms at which R¹¹ and R¹² are placed; R¹⁴ is hydrogen or hydroxy, or R¹⁴ designates, together with R¹⁵, an additional bond between the carbon atoms at which R¹⁴ and R¹⁵ are placed; R¹⁵ is selected from the group consisting of hydrogen, halogen, lower alkyl, methylene, hydroxy, lower alkoxy, oxo and a group of the formula=NOR³² wherein R³² is hydrogen or lower alkyl, or R¹⁵ designates, together with R¹⁴ an additional bond between the carbon atoms at which R¹⁵ and R¹⁴ are placed; R¹⁶ is selected from the group consisting of hydrogen, halogen, lower alkyl, methylene, hydroxy, lower alkoxy, oxo and a group of the formula=NOR³⁴ wherein R³⁴ is hydrogen or lower alkyl, or R¹⁶ designates, together with R¹⁷, an additional bond between the carbon atoms at which R¹⁶ and R¹⁷ are placed; R¹⁷ is hydrogen or hydroxy, or R¹⁷ designates, together with R¹⁶, an additional bond between the carbon atoms at which R¹⁷ and R¹⁶ are placed; R²⁰ is selected from the group consisting of hydrogen, lower alkyl and hydroxymethyl, or R²⁰ and R′²⁰ together designate methylene or oxo; R′²⁰ is hydrogen, halogen, lower alkyl or hydroxy, R′²² is hydrogen, hydroxy or oxo; R²² represents a group of the formula:
—C(²³) (R′²³)—C(R²⁴) (R′²⁴)—A(R²⁵) (R′²⁵) (R″²⁵),
wherein R²³ and R′²³ are both hydrogen, or one of R²³ and R′²³ is hydrogen while the other is halogen, hydroxy or methoxy, or R²³ and R′²³ together designates oxo, R′²⁴ is hydrogen when R²⁴ is different from oxo, and R′²⁴ is absent when R²⁴ is oxo; and A is a carbon atom or a nitrogen atom; and when A is a carbon atom, R²⁵ is selected from the group consisting of hydrogen, hydroxy and halogen; and R²⁴ is selected from the group consisting of hydrogen, halogen, hydroxy, lower alkyl, methylene and oxo, or R²⁵ designates, together with R²⁴, an additional bond between the carbon atoms at which R²⁴ and R²⁵ are placed; R′²⁵ is selected from the group consisting of lower alkyl, trifluoromethyl and C₃-C₆ cycloalkyl; R″²⁵ is selected from the group consisting of lower alkyl, hydroxy(lower alkyl), halogen(lower alkyl) containing up to three halogen atoms, methoxymethyl, acetoxymethyl, and C₃-C₆ cycloalkyl, or R′²⁵ and R″²⁵, together with the carbon atom at which they are placed, form a C₃-C₆ cycloalkyl ring; and when A is a nitrogen atom, R²⁵ designates a lone pair of electrons; and R²⁴ is selected from the group consisting of hydrogen, hydroxy, lower alkyl, cyano and oxo; and R′²⁵ and R″²⁵ are, independently, lower alkyl or C₃-C6 cycloalkyl; or R²² is phenyl, toluyl, hydroxyphenyl, cyclopentyl, cyclohexyl, isobutyl or cyclohexyloxycarbonylmethyl; and esters, salts, active metabolites and prodrugs thereof.

5. A compound of the formula Ib 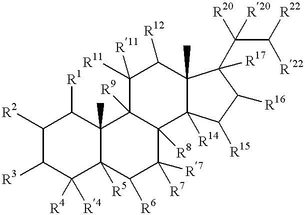
wherein R¹ is hydrogen, halogen, methyl, hydroxy or oxo; R² is selected from the group consisting of hydrogen, hydroxy, lower alkyl, vinyl, lower alkoxy and halogen, or R² designates, together with R³, an additional bond between the carbon atoms at which R² and R³ are placed; R³ is hydrogen or lower alkyl; or R³ designates, together with R⁴, an additional bond between the carbon atoms at which R³ and R⁴ are placed; or R³ designates, together with R², an additional bond between the carbon atoms at which R² and R³ are placed; R⁴ and R′⁴, which are different or identical provided that they are not both hydroxy, are selected from the group consisting of hydrogen, halogen, hydroxy and lower alkyl which may be substituted by halogen, hydroxy or cyano, or wherein R⁴ and R′⁴ together designate methylene or oxo or, together with the carbon atom to which they are bound, form a cyclopropane ring, a cyclopentane ring, or a cyclohexane ring; or R⁴, R′⁴ and R⁵ together designate an additional bond between the carbon atoms in the 4 and 5 position, R⁵ is hydrogen, halogen or hydroxy, or R⁵ designates, together with R⁶, an additional bond between the carbon atoms at which R⁵ and R⁶ are placed; R⁶ is hydrogen, hydroxy, halogen or oxo, or R⁶ designates, together with R⁵ or R⁷, an additional bond between the carbon atoms at which R⁶ and R⁵ or R⁷ are placed; R⁷ is selected from the group consisting of hydrogen, hydroxy, methoxy, acyloxy, halogen and lower alkyl, or R⁷ designates, together with R⁶ or R⁸, an additional bond between the carbon atoms at which R⁷ and R⁶ or R⁸ are placed; and R′⁷ is hydrogen or, if R⁷ is lower alkyl, R′⁷ is hydrogen or hydroxy; or R⁷ and R′⁷ together designates oxo, methylene or a group of the formula=NOR³⁶ wherein R³⁶ is hydrogen or lower alkyl; R⁸ is hydrogen, hydroxy or halogen, or R⁸ designates, together with R⁷, R⁹ or R¹⁴, an additional bond between the carbon atoms at which R⁸ and R⁷, R⁹or R¹⁴ are placed; R⁹ is hydrogen, hydroxy or halogen, or R⁹ designates, together with R⁸ or R¹¹, an additional bond between the carbon atoms at which R⁹ and R⁸ or R¹¹ are placed; R¹¹ is selected from the group consisting of hydrogen, hydroxy, methoxy, acyloxy, halogen and lower alkyl, or R¹¹ designates, together with R⁹ or R¹², an additional bond between the carbon atoms at which R¹¹ and R⁹ or R¹² are placed; and R′¹¹ is hydrogen or, if R¹¹ is lower alkyl, R′¹¹ is hydrogen or hydroxy; or R¹¹ together with R′¹¹ is oxo, methylene or a group of the formula=NOR³⁷ wherein R³⁷ is hydrogen or lower alkyl; R¹² is selected from the group consisting of hydrogen, halogen, lower alkyl, methylene, hydroxy, lower alkoxy, acyloxy, oxo and a group of the formula=NOR³³ wherein R³³ is hydrogen or lower alkyl, or R¹² designates, together with R¹¹, an additional bond between the carbon atoms at which R¹¹ and R¹² are placed; R¹⁴ is hydrogen or hydroxy, or R¹⁴ designates, together with R¹⁵, an additional bond between the carbon atoms at which R¹⁴ and R¹⁵ are placed; R¹⁵ is selected from the group consisting of hydrogen, halogen, lower alkyl, methylene, hydroxy, lower alkoxy, oxo and a group of the formula=NOR³² wherein R³² is hydrogen or lower alkyl, or R¹⁵ designates, together with R¹⁴ an additional bond between the carbon atoms at which R¹⁵ and R¹⁴ are placed; R¹⁶ is selected from the group consisting of hydrogen, halogen, lower alkyl, methylene, hydroxy, lower alkoxy, oxo and a group of the formula=NOR³⁴ wherein R³⁴ is hydrogen or lower alkyl, or R¹⁶ designates, together with R¹⁷, an additional bond between the carbon atoms at which R¹⁶ and R¹⁷ are placed; R¹⁷ is hydrogen or hydroxy, or R¹⁷ designates, together with R¹⁶, an additional bond between the carbon atoms at which R¹⁷ and R¹⁶ are placed; R²⁰ is selected from the group consisting of hydrogen, lower alkyl and hydroxymethyl, or R²⁰ and R′²⁰ together designate methylene or oxo; R′²⁰ is hydrogen, halogen, lower alkyl or hydroxy, R′²² is hydrogen, hydroxy or oxo; R²² represents a group of the formula:
—C(R²³) (R′²³)—C(R²⁴) (R′²⁴)—A(R²⁵) (R′²⁵) (R″²⁵),
wherein R²³ and R′²³ are both hydrogen, or one of R²³ and R′²³ is hydrogen while the other is halogen, hydroxy or methoxy, or R²³ and R′²³ together designates oxo, R′²⁴ is hydrogen when R²⁴ is different from oxo, and R′²⁴ is absent when R²⁴ is oxo; and A is a carbon atom or a nitrogen atom; and when A is a carbon atom, R²⁵ is selected from the group consisting of hydrogen, hydroxy and halogen; and R²⁴ is selected from the group consisting of hydrogen, halogen, hydroxy, lower alkyl, methylene and oxo, or R²⁵ designates, together with R²⁴, an additional bond between the carbon atoms at which R²⁴ and R²⁵ are placed; R′²⁵ is selected from the group consisting of lower alkyl, trifluoromethyl and C₃-C₆ cycloalkyl; R″²⁵ is selected from the group consisting of lower alkyl, hydroxy(lower alkyl), halogen(lower alkyl) containing up to three halogen atoms, methoxymethyl, acetoxymethyl, and C₃-C₆ cycloalkyl, or R′²⁵ and R″²⁵, together with the carbon atom at which they are placed, form a C₃-C₆ cycloalkyl ring; and when A is a nitrogen atom, R²⁵ designates a lone pair of electrons; and R²⁴ is selected from the group consisting of hydrogen, hydroxy, lower alkyl, cyano and oxo; and R′²⁵ and R″²⁵ are, independently, lower alkyl or C₃-C₆ cycloalkyl; or R²² is phenyl, toluyl, hydroxyphenyl, cyclopentyl, cyclohexyl, isobutyl or cyclohexyloxycarbonylmethyl; and esters, salts, active metabolites and prodrugs thereof.

6. A method of regulating meiosis comprising administering to a subject in need thereof, an effective amount of a compound of claim 5.

7. A method of regulating meiosis in a mammalian germ cell comprising administering to a germ cell in need thereof an effective amount of a compound of claim 5.

8. The method according to claim 7 wherein the compound is administered to the germ cell by administering it to a mammal hosting the germ cell.

9. The method according to claim 7 wherein the germ cell is an oocyte.

10. The method according to claim 7 wherein the compound is administered to an oocyte ex vivo or in vitro.

11. The method according to claim 7 wherein the germ cell is a male germ cell.

12. The method according to claim 7 whereby mature male germ cells are produced by administering a compound of claim 5 to testicular tissue in vivo, ex vivo or in vitro.

13. A pharmaceutical composition for regulating meiosis in a mammalian germ cell, the composition comprising as an active ingredient an effective amount of a compound of claim 5 together with a pharmaceutically acceptable carrier.
